(19) United States
(12) Patent Application Publication
(10) Pub. No.: US 2026/0306051 A1
(43) Pub. Date: Oct. 8, 2026

(54) Method and System for Estimating Ultrasound Probe Pose in Laparoscopic Ultrasound Procedures

(71) Applicant: EDDA TECHNOLOGY, INC., PRINCETON, NJ (US)

(72) Inventors: Xiao Qi, Edison, NJ (US); Xiaonan Zang, Princeton, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 19/481,799

(22) PCT Filed: May 2, 2024
(86) PCT No.: PCT/US2024/027475

§ 371 Date: Nov. 5, 2025

Related U.S. Application Data
(60) Provisional application No. 63/464,282, filed on May 5, 2023.

(51) Int. Cl. A61B 8/08; A61B 1/313; A61B 8/00; A61B 8/12; G06T 7/73

(52) U.S. Cl. CPC A61B 8/085; A61B 1/3132; A61B 8/12; A61B 8/4245; G06T 7/73; G06T 2207/10132; G06T 2207/20081

(57) ABSTRACT

A method and system for estimating a 3D pose of an ultrasound probe based on 2D ultrasound images acquired. During a surgery, 3D probe poses are projected onto a 2D laparoscopic image at corresponding 2D locations. An ultrasound probe is placed at/near a selected one of the 3D probe poses to acquire 2D ultrasound images, from which anatomical structures (AS) are detected and used to generate AS masks (ASMs). 3D probe poses of the ultrasound probe are estimated using an ASM-pose mapping model based on the ASMs and are used to generate an optimal 3D probe pose estimate. A method and system for estimating a 3D pose of an ultrasound probe based on 2D ultrasound images acquired. During a surgery, 3D probe poses are projected onto a 2D laparoscopic image at corresponding 2D locations. An ultrasound probe is placed at/near a selected one of the 3D probe poses to acquire 2D ultrasound images, from which anatomical structures (AS) are detected and used to generate AS masks (ASMs). 3D probe poses of the ultrasound probe are estimated using an ASM-pose mapping model based on the ASMs and are used to generate an optimal 3D probe pose estimate.

### CROSS REFERENCE TO RELATED APPLICATION

The present application claims priority benefit of the filing date of U.S. Provisional Patent Application No. 63/464,282, filed May 5, 2023, which is herein incorporated by reference in its entirety.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including the medical industry, where large volume of image data, capturing anatomical information of a patient, may be processed by computers to identify anatomical structures of interest (e.g., organs, bones, blood vessels, or abnormal nodule), obtain measurements for each object of interest (e.g., dimension of a nodule growing in an organ), and visualize relevant features (e.g., three-dimensional (3D) visualization of an abnormal nodule).

Such information may be used for a wide variety of purposes. For example, a 3D model may be constructed for an organ (e.g., a liver in terms of its volume, shape, or size along different dimensions) or for different parts of the organ (e.g., a nodule growing inside a liver, vessel structures inside and near the organ). An example of a 3D model 100 for a liver is shown in FIG. 1A and a 3D vessel tree model 110 constructed based on vessels detected inside of a liver is shown in FIG. 1B. Such constructed 3D models may also be used for different purposes, such as diagnosis, presurgical planning, and in-surgical navigation.

FIG. 1C shows a laparoscopic ultrasound (LUS) surgical setting where a patient 130 on top of a surgical table 120 is undergoing a laparoscopic procedure. A laparoscopic camera 140 is inserted into the patient's body and can be controlled to reach a position where it can capture video images 150 of the scene observable near a surgical instrument (not shown).

While a user such as a surgeon is manipulating the surgical tool to perform a surgical action, the video images acquired by the laparoscopic camera 140 about what is visible near the surgical instrument may provide visual guidance to the surgeon. In addition, an ultrasound probe 160 may also be inserted into the patient body during the surgery and is used to scan to “see” what is beneath an organ. For instance, in a liver resection surgery, prior to a surgeon to cut open the liver, an ultrasound probe may be used to help to “see” what is inside of the liver in order to assist the surgeon to determine how to cut open the liver. An exemplary ultrasound image formed based on information acquired by an ultrasound probe 160 is shown in 170 in FIG. 1C.

An ultrasound probe operates by sending out beams of sound waves, which travel alone a path to a plane and get reflected back by object at the plane, and ultrasound images are generated based on the reflected sound waves. In general, what an ultrasound probe “sees” corresponds to the information of a slice in space or a plane, as illustrated in FIG. 1D, where the ultrasound probe scans an organ 180 and what is “observable” is a slice of information from the plane 190. As such, what is captured by an ultrasound probe may be limited or incomplete. One example is illustrated in FIG. 1E, wherein an ultrasound image 195 generated based on information acquired from a plane 190 shows a vessel structure 197 inside an organ, which is visible because it lies on plane 190 reached by the sound wave beams emitted by the ultrasound probe. As compared with the full 3D vessel tree 110 of the organ, shown in FIG. 1B, the vessel structure 197 corresponds to only a portion of the 3D vessel tree 110 in a slice in space corresponding to plane 190 as marked in FIG. 1B.

Such partial information may make it difficult for a surgeon to determine what corresponds to the vessels observed in an ultrasound image and make an operational decision accordingly. For example, in some surgeries on an organ such as a liver, some blood vessels may need first to be pushed away or clapped to temporarily stop the blood flow. In those situations, if what is seen cannot inform the surgeon which vessels they correspond to, the probed ultrasound images may cause confusion rather than help.

Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for estimating a 3D pose of an ultrasound probe based on 2D ultrasound images acquired. During a surgery, 3D probe poses are projected onto a 2D laparoscopic image at corresponding 2D locations. An ultrasound probe is placed at/near a selected one of the 3D probe poses to acquire 2D ultrasound images, from which anatomical structures (AS) are detected and used to generate AS masks (ASMs). 3D probe poses of the ultrasound probe are estimated using an ASM-pose mapping model based on the ASMs and are used to generate an optimal 3D probe pose estimate.

In a different example, a system is disclosed for estimating a 3D pose of an ultrasound probe based on 2D ultrasound images acquired. which includes an ultrasound probe pose projector and an ultrasound-based 3D anatomical structure visualizer. The former is for projecting, during a surgery, one or more three-dimensional (3D) probe poses onto a 2D laparoscopic image at corresponding 2D probe locations. An ultrasound probe is placed at/near a selected one of the 3D probe poses to acquire 2D ultrasound images. The ultrasound-based 3D anatomical structure visualizer is for detecting anatomical structures (AS) from the 2D ultrasound images to generate AS masks (ASMs) and estimating, based on the ASMs, 3D probe poses of the ultrasound probe using an ASM-pose mapping model. The estimated 3D probe poses are used to generate an optimal 3D probe pose estimate.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for estimating a 3D pose of an ultrasound probe based on 2D ultrasound images acquired. The information on the medium, when read by the machine, causes the machine to perform various steps. 3D probe poses are projected, during a surgery, onto a 2D laparoscopic image at corresponding 2D locations. An ultrasound probe is placed at/near a selected one of the 3D probe poses to acquire 2D ultrasound images, from which anatomical structures (AS) are detected and used to generate AS masks (ASMs). 3D probe poses of the ultrasound probe are estimated using an ASM-pose mapping model based on the ASMs and are used to generate an optimal 3D probe pose estimate.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIGS. 1A-1B show an exemplary 3D model of an organ and 3D vessel tree model for vessels inside the organ;

FIG. 1C shows a typical LUS surgical setting;

FIGS. 1D-1E illustrate characteristics of an ultrasound probe and information it acquires;

FIG. 2 depicts an exemplary high level system diagram of a system for estimating a 3D ultrasound probe pose (USPP) and visualizing anatomical structures (AS) detected based on the 3D USPP, in accordance with an embodiment of the present teaching;

FIG. 3A shows 3D USPP selected by a user, in accordance with an embodiment of the present teaching;

FIGS. 3B-3C illustrate discrete mappings from anatomical structure masks (ASMs) to 3D USPPs automatically generated as training data for machine learning, in accordance with embodiments of the present teaching;

FIG. 3D shows an exemplary ASM obtained based on vessels detected from a 2D ultrasound image, in accordance with an embodiment of the present teaching;

FIG. 3E illustrates an ASM-pose mapping model created via machine learning based on discrete ASM-pose mapping data, in accordance with an embodiment of the present teaching;

FIG. 4A is a flowchart of a pre-surgery process of a 3D USPP-based AS visualization system to generate an ASM-pose mapping model via selected ultrasound probe poses with respect to a 3D organ model, in accordance with an embodiment of the present teaching;

FIG. 4B is a flowchart of an in-surgery process of a 3D USPP-based AS visualization system to estimate a 3D ultrasound probe pose in an LUS procedure based on an ASM-pose mapping model, in accordance with an embodiment of the present teaching;

FIG. 5A depicts an exemplary high level system diagram of an ASM-pose mapping model generator, in accordance with an embodiment of the present teaching;

FIG. 5B is a flowchart of an exemplary process of an ASM-pose mapping model generator, in accordance with an embodiment of the present teaching;

FIG. 6A depicts an exemplary high level system diagram of a pose-based 2D ASM determiner, in accordance with an embodiment of the present teaching;

FIG. 6B is a flowchart of an exemplary process of a pose-based 2D ASM determiner, in accordance with an embodiment of the present teaching;

FIG. 7A illustrates 2D projections of exemplary 3D ultrasound probe poses selected with respect to a 3D model, in accordance with an embodiment of the present teaching;

FIG. 7B depicts an exemplary high level system diagram of an ultrasound-based 3D anatomical structure visualizer, in accordance with an embodiment of the present teaching;

FIG. 7C is a flowchart of an exemplary process of an ultrasound-based 3D anatomical structure visualizer, in accordance with an embodiment of the present teaching;

FIG. 7D illustrates an exemplary operation to estimate a 3D USPP based on ASMs extracted from 2D ultrasound images, in accordance with an embodiment of the present teaching;

FIG. 8 is an illustrative diagram of an exemplary mobile device architecture that may be used to realize a specialized system implementing the present teaching in accordance with various embodiments; and

FIG. 9 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations for a 3D USPP based AS visualization system in an LUS procedure. An LUS procedure may be used in different surgeries such as laparoscopic liver resection. The use of an ultrasound probe may provide views of anatomical structures beneath surface of an organ such as vessels and/or tumors. Prior to a surgery, 3D organ models may be constructed based on 3D medical images obtained via scans such as computerized tomography (CT) or magnetic resonance imaging (MRI). Ultrasound images acquired by an ultrasound probe are generally noisy and are with only partial information. As such, ultrasound image in general cannot provide adequate information to reveal anatomical structures to provide effective visual guide during a surgery.

The present teaching discloses method and system for estimating a 3D probe pose of an ultrasound probe (USPP) based on partial information observed in laparoscopic and ultrasound images. The estimated 3D USPP may then be utilized to determine how to visualize 3D anatomical structures corresponding to what is observed in 2D images. Specifically, the estimated 3D USPP is used to determine an appropriate perspective with respect to the 3D organ models so that 3D anatomical structures modeled in the 3D organ models as viewed from the perspective can be visualized to allow a user to see what corresponds to the limited content observable from 2D ultrasound images. To enable estimation of a 3D USPP, the method and system according to the present teaching involves both pre-surgical and in-surgical operations. In a pre-surgical planning stage, various 3D USPP may be specified, each of which may be used to determine a perspective to view, via a simulated ultrasound probe, the 3D organ models to create a 2D simulated ultrasound image with anatomical structures as captured by the ultrasound probe.

The anatomical structures in each simulated ultrasound image may be identified so that an anatomical structure mask (ASM) may be generated. An ASM may correspond to a binarized 2D image with pixels on an anatomical structure being in one state (e.g., state 1) and everywhere else being in another state (e.g., state 0). With a set of specified 3D probe poses and their corresponding ASMs, an ASM-pose mapping model may be created via machine learning for predicting a 3D probe pose based on a 2D ASM obtained based on a 2D ultrasound image. The ASM-pose mapping model created this way may be used subsequently during a surgery by obtaining a 2D ASM based on an ultrasound image acquired in real time and predicting candidate 3D USPPs with, e.g., respective probabilities or confidence scores.

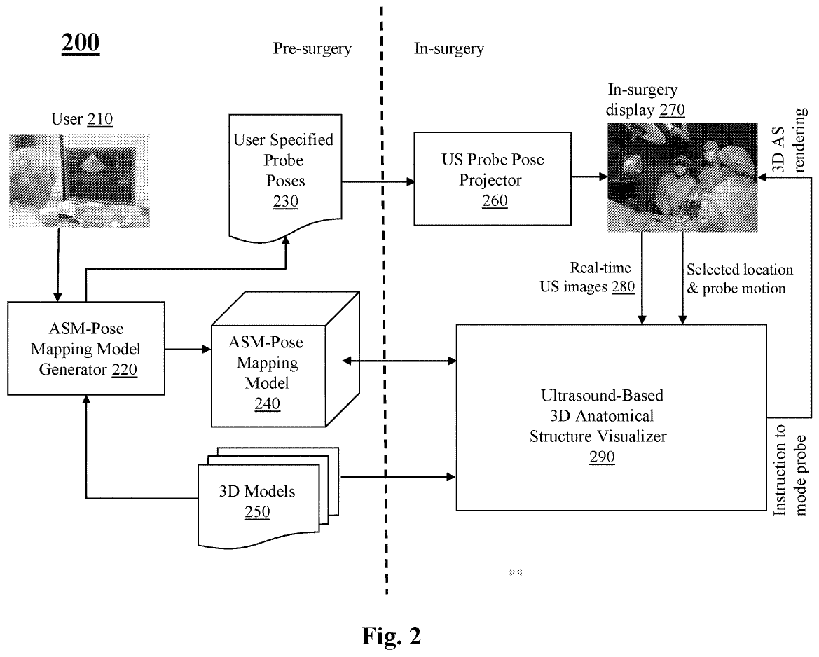

During a LUS procedure, 2D ultrasound images may be acquired by an ultrasound probe manipulated by a user. Such 2D ultrasound images may be processed to identify anatomical structures to obtain corresponding ASMs. Utilizing the ASM-pose mapping model created pre-surgery, for each ASM derived from a 2D ultrasound image, the ASM-pose mapping model predicts multiple candidate 3D probe poses with probabilities, which may then be used for optimization to identify a best estimated 3D USPP. The estimated 3D USPP may then be used to determine a perspective with respect to the 3D organ models so that 3D anatomical structures in the 3D organ models as viewed from the perspective correspond to the anatomical structures partially visible in 2D ultrasound images. The 3D anatomical structures so identified may be rendered to the user in a perspective consistent with what is partially seen in 2D ultrasound images yet with a 3D view of the full anatomical structures. Details of the pre-surgical and in-surgery operations to achieve estimation of 3D ultrasound probe pose and visualization of 3D anatomical structures determined based on the estimated 3D probe pose are provided herein with references to FIGS. 2A-7C .

FIG. 2 depicts an exemplary high level system diagram of a 3D USPP based AS visualization system 200, in accordance with an embodiment of the present teaching. System 200 includes two portions, one being a pre-surgery portion and the other being an in-surgery portion. The pre-surgery portion comprises an ASM-pose mapping model generator 220 that renders 3D models 250 for a target organ to a user 210, interacts with the user 210 to receive user specified probe poses 230 defined with respect to the 3D models 250. Based on those user specified probe poses 230, the ASM-pose mapping model generator 220 creates an ASM-pose mapping model 240. In some embodiments, the ASM-pose mapping model 240 is obtained via machine learning based on training data that is created based on the user specified probe poses 230.

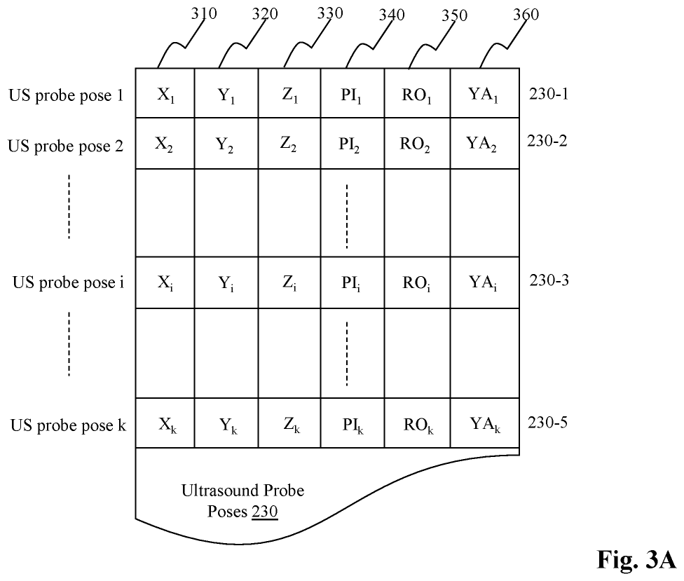

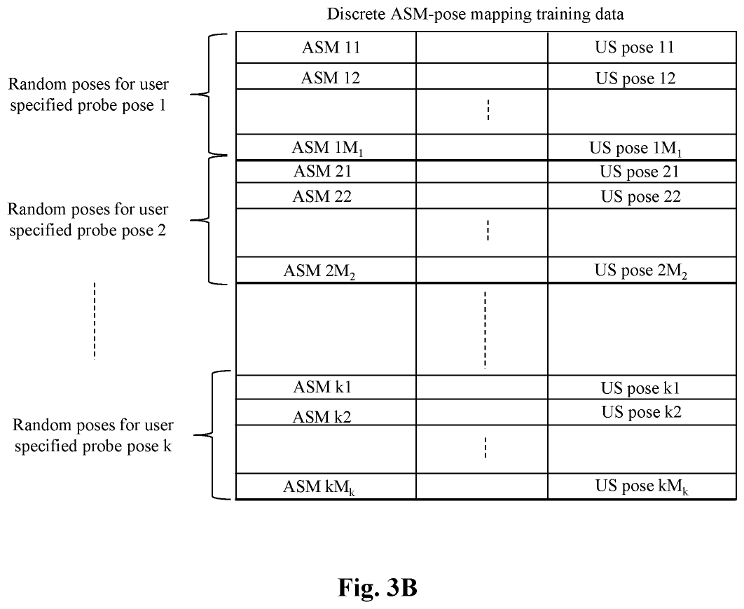

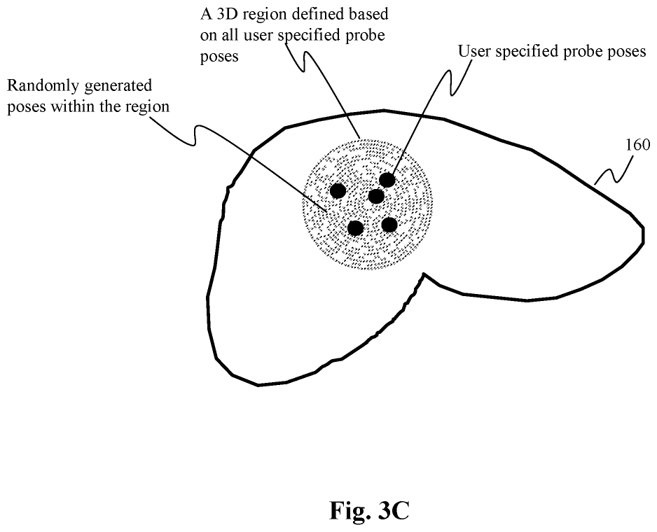

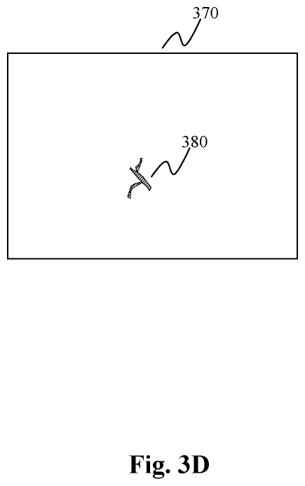

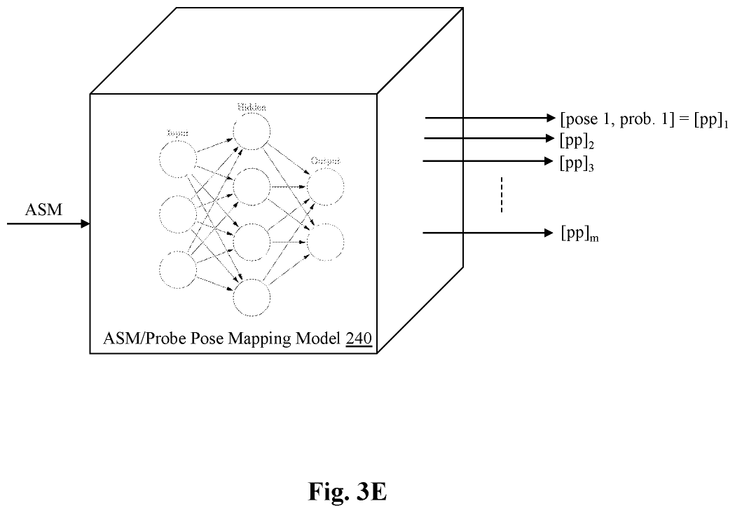

The in-surgery portion comprises an ultrasound (US) probe pose projector 260, one or more in-surgery displays 270, and an ultrasound-based 3D anatomical structure (AS) visualizer 290. During a LUS procedure, a laparoscopic camera acquires 2D laparoscopic images. The US probe pose projector 260 is provided for projecting the user specified 3D probe poses 230 onto the 2D laparoscopic images as 2D projected locations to guide a user as to where to place an ultrasound probe during the surgery. Based on the projected 2D probe locations, the user may select to place the ultrasound probe at or near one of these 2D probe locations. The ultrasound probe, once placed, acquires real time ultrasound (US) images 280. The selected 2D probe location and the real time US images 280 are then sent to the ultrasound-Based 3D AS visualizer 290, which utilizes the ASM-pose mapping model 240 to estimate the 3D ultrasound probe pose or 3D USPP of the ultrasound probe that corresponds to the selected 2D probe location. Estimating the 3D USPP is needed for the following reason. Due to deformation and/or inaccuracy in registration, the 2D probe locations projected from the user specified probe poses 230 to 2D images may not accurately correspond to the 3D user specified probe poses 230. Given that, the actual 3D USPP corresponding to the selected 2D probe location has to be estimated. Details related to the ASM-pose mapping model generator 220 and the ultrasound-based 3D anatomical structure visualizer 290 will be provided with reference to FIGS. 3A-7C .

As discussed herein, the ASM-pose mapping model 240 is obtained prior to a surgery. To do so, the 3D models 250 for the target organ may first be visualized to a user. The visualized 3D models may be manipulated by the user, e.g., via rolling, transposing, etc. to see the 3D models in different views. The user may select any 3D location in the visualized 3D models as a simulated ultrasound probe pose, which defines a perspective to view the 3D models. FIG. 3A illustrates exemplary user specified 3D US probe poses and representations thereof, in accordance with an embodiment of the present teaching. As shown in FIG. 3A, there are k user specified 3D probe poses, i.e., pose 1 230-1, pose 2 230-2, . . . , pose i 230-3, . . . , and pose k 230-5. Such user specified probe poses may be represented based on, e.g., parameters in their respective 6 degrees of freedom, including, e.g., 3D coordinate defined in X axis 310, in Y axis 320, in Z axis 330, as well as pitch (PI) 340, roll (RO) 350, and yaw (YA) 360.

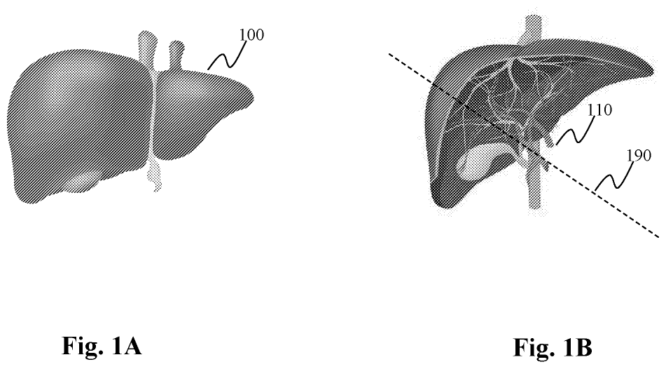

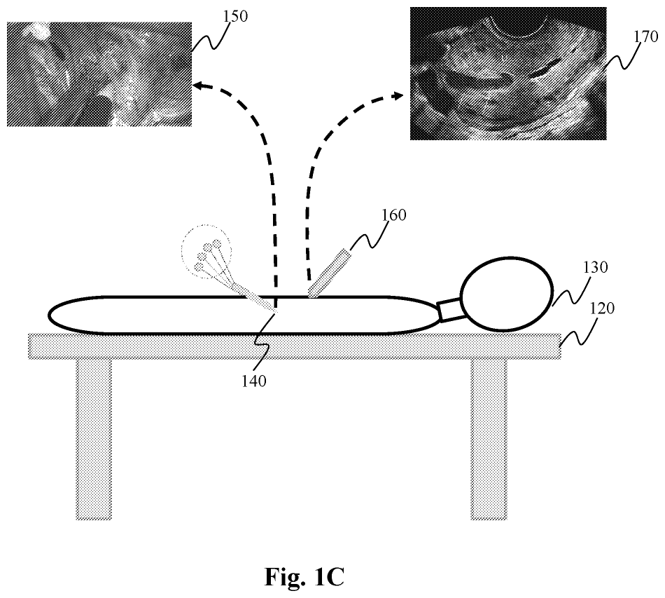

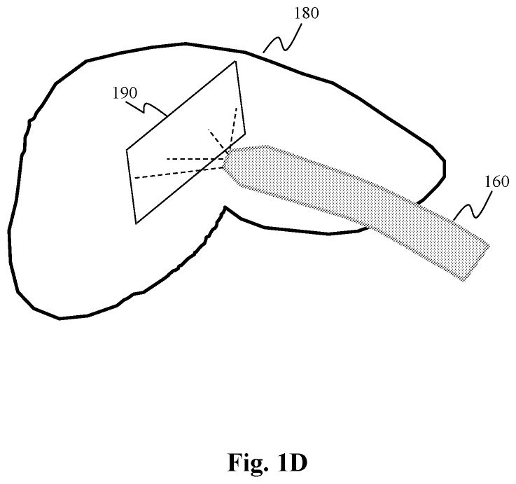

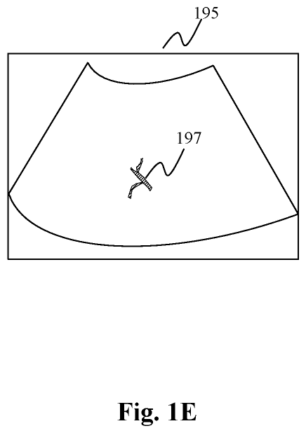

Each user specified probe pose defines a perspective with respect to the visualized 3D models and can be applied to generate a simulated ultrasound image that can be captured by a ultrasound probe. One example is illustrated in FIG. 1D, where an ultrasound probe 160, once positioned at a particular location with a perspective with respect to an organ 180, yields a view 190 of the organ determined via the perspective. That is, each user specified probe pose may be used to determine a slice 190 in the 3D models, which may be used to generate a 2D ultrasound image with information on the slice 190. For each of such 2D slice images, anatomical structures as seen therein may be detected and a corresponding ASM may be generated. In this way, an ASM obtained for a 2D slice image corresponds to a user specified probe pose that yields a ASM-3D USPP pair or a mapping from an ASM to a 3D probe pose.

To construct the ASM-pose mapping model 240, the mappings from ASMs generated based on 2D slice images to corresponding user specified probe poses may be used to map an ASM obtained based on real time ultrasound image to an estimated 3D USPP. In some embodiments, the pairs may be used as seeds to generate additional mappings as training data to machine learn the ASM-pose mapping model 240. In some embodiments, the additional mappings may be created in the following way. With respect to each user specified probe pose, a series of probe poses may be randomly generated in a pre-determined vicinity around each user specified pose. For example, with respect to a user specified probe pose, 10,000 probe poses may be randomly generated within a sphere centered at the user specified probe pose and with a given diameter. FIG. 3B illustrates an example, where for user specified probe pose 1, a first set of random poses is generated, for user specified probe pose 2, a second set of random poses are generated, . . . , for user specified probe pose k, a kth set of random poses are generated. For each of the 3D probe pose, either user specified or randomly generated, a 2D slice image may be obtained via the 3D models and an ASM may be generated accordingly. In this way, many ASM-pose mappings may be created as shown in FIG. 3B, e.g., ASMᵢⱼ-->US poseᵢⱼ, where 1,1<i,j<k, Mₖ. This operation produces discrete ASM-pose mapping pairs, which may be used as a discrete mapping model 240 or used as training data for training, e.g., via machine learning, the ASM-pose mapping model 240, that is capable of extrapolating the training data via learning the mapping based on any ASM, including the ones that are not included in the training data.

In a different embodiment, the additional random probe poses generated based on user specified probe poses 230 may be achieved according to a different scheme. FIG. 3C illustrates a scheme where additional random poses are generated within a 3D region defined based on user specified probe poses, in accordance with different embodiments of the present teaching. In this scheme, based on the multiple user specified probe poses (the black dots in FIG. 3C), a 3D region is obtained according to some specified criteria (e.g., a 3D region encompassing all user specified probe poses). Additional random poses are then randomly generated within this 3D region (see smaller dots within the sphere). In some situations, a density criterion may also be specified so that the number of additional probe poses is to meet the density criterion. Once the additional random probe poses are generated, each is used to yield a 2D slice image and an ASM to generated a mapping from the ASM to the probe pose. In this way, a training data set with ASM-->probe pose mappings can be generated and used for training the ASM-pose mapping model 240.

In generating ASM-pose mappings as training data, as the ASMs are obtained based on 2D slice images and each 2D slice image may include only partial views of certain anatomical structure, such detected ASMs simulates what is to be seen by an ultrasound probe during a surgery. FIG. 3D shows an exemplary ASM obtained based on vessels partially visible from a 2D ultrasound image. In FIGS. 3D, 370 represents a 2D image and 380 represents the partial anatomical structure (e.g., vessels) visible in the 2D image. It is partial because the 2D image represents a slice of the organ, either created during the simulation process as discussed herein or an actual 2D ultrasound image acquired during a surgery. Given that, a ASM (e.g., binary image for 370, wherein pixels on the vessel structure have 1's and everywhere else have 0s) obtained from 2D image 370 may reveal what is visible on that slice given a ultrasound probe at a 3D USPP. Thus, each 3D probe pose (either simulated or actual) yields an ASM generated from a 2D image having a partial view of some anatomical structure. The mappings may be used to derive the ASM-pos mapping model 240, which may be used during a surgery to predict a 3D USPP based on a given ASM, obtained from a real time ultrasound image. That is, the predicted 3D USPP is a 3D location of the ultrasound probe that allows a user to see what is visible in the real time ultrasound image.

FIG. 3E illustrates the ASM-pose mapping model 240 generated via machine learning based on discrete ASM-pose mapping training data, in accordance with an embodiment of the present teaching. In some embodiments, the ASM-pose mapping model 240 may be implemented using an artificial neural network which may be trained via, e.g., deep learning, to predict candidates 3D USPPs based on a given ASM generated based on a 2D ultrasound image. As shown, in some embodiments, each of the predicted 3D USPPs may be output with a corresponding probability to indicate, e.g., the confidence in the prediction.

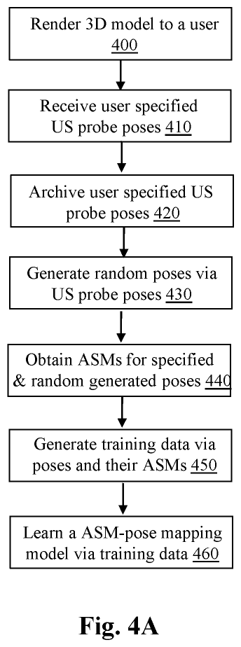

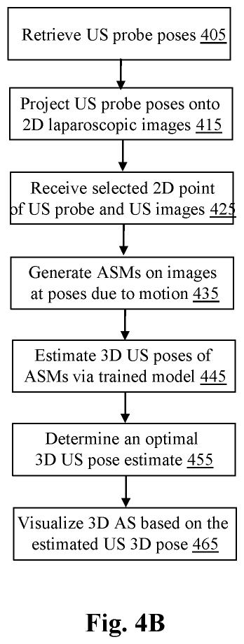

FIG. 4A is a flowchart of an exemplary pre-surgery process for generating the ASM-pose mapping model 240, in accordance with an embodiment of the present teaching. To allow a user to choose user specified probe poses, the ASM-pose mapping model generator 220 renders, at 400, the 3D models 250 for a target organ. As discussed herein, the user may manipulate the rendering and determine selected simulated probe poses. When information on user specified probe poses is received at 410, the selected probe poses 230 are archived at 420. In some embodiments, additional random probe poses may be generated, at 430, based on the user specified probe poses according to some generation scheme as disclosed herein. Based on the simulated probe poses, either specified or randomly generated, their ASMs are obtained, at 440, from 2D slice images created based on the 3D models 250 according to the simulated probe poses. The mappings from ASMs to the simulated probe poses are then used to generate, at 450, training data, which are then used to train, via machine learning at 460, the ASM-pose mapping model 240.

FIG. 4B is a flowchart of an exemplary in-surgery process for visualizing 3D anatomical structures captured by an ultrasound probe and determined based on a predicted 3D USPP based on an ASM-pose mapping model, in accordance with an embodiment of the present teaching. During a surgery, the US probe pose projector 260 retrieves, at 405, archived user specified probe poses 230 and projects, at 415, them onto 2D laparoscopic images. The projection may be based on a registration between the 3D models 250 and the 2D laparoscopic images. As noted herein, such registration is frequently inaccurate and, hence, unreliable (due to various reasons such as deformation or limited field of view of the laparoscopic images). However, such projected user specified probe poses in laparoscopic images may be useful to provide a guidance to a user to determine where to position the ultrasound probe. The accuracy is to be achieved via estimating the actual 3D USPP based on the ASP-pose mapping model obtained according to the present teaching.

Based on the user specified probe poses projected in 2D laparoscopic images, a user may select one of these poses (e.g., by clicking on one projected pose) to place the ultrasound probe at or near the selected user specified 3D probe pose. Information on the selected 2D projected point may be received from the user and used to deploy the ultrasound probe at the selected 3D pose. With the ultrasound probe so deployed, the ultrasound-based 3D anatomical structure visualizer 290 receives, at 425, real time US images. The user selected probe pose as projected and appearing in 2D laparoscopic images may serve as an initial estimate of the actual 3D USPP of the ultrasound probe. To enhance the accuracy of the estimate, the user may be instructed to introduce motion of the probe via manipulation, such as rolling the probe in a clockwise or counterclockwise directions. Such motion may produce a series of 2D ultrasound images, each of which may reveal varying information. Based on such series of 2D ultrasound images, corresponding ASMs may be generated at 435 by detecting the anatomical structures observed in ultrasound images and creating a binarized representation of the detected anatomical structures. Taking the ASMs generated based on 2D ultrasound images, candidate 3D USPPs may be estimated, at 445, using the ASM-pose mapping model 240. An optimal 3D USPP estimate may then be determined, at 455, via some optimization scheme to be explained below. The optimized 3D USPP estimate may then be used to determine a perspective to look into the 3D models 240 so that anatomical structures on a slice (determined based on the perspective and the operational parameters of the ultrasound probe) may be visualized, at 465, to the user to provide an improved visual guidance on what corresponds to the information in the ultrasound images.

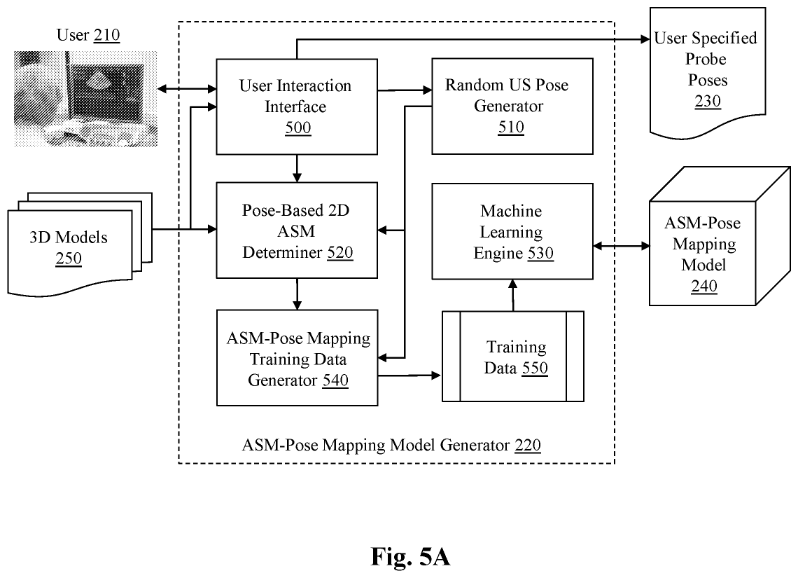

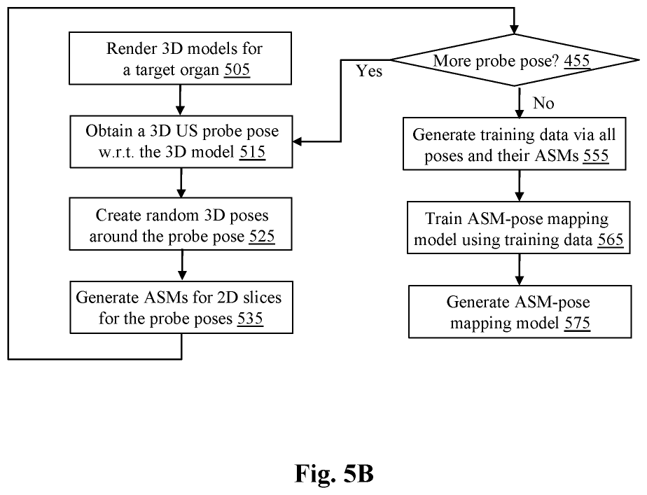

Below, details of the ASM-pose mapping model generator 220 and the ultrasound-based 3D anatomical structure visualizer 290 are disclosed. FIG. 5A depicts an exemplary high level system diagram of the ASM-pose mapping model generator 220, in accordance with an embodiment of the present teaching. As discussed herein, the ASM-pose mapping model 240 is for mapping an ASM obtained from a 2D ultrasound image (either simulated or actual) to a 3D USPP. The ASM-pose mapping model 240 may be a discrete model as a look up table (as shown in FIG. 3B) or is obtained via machine learning based on training data created from the discrete mapping pairs as shown in FIG. 3B. In some exemplary embodiments, the ASM-pose mapping model generator 220 comprises a user interaction interface 500, a random US pose generator 510, a pose based 2D ASM determiner 520, an ASM-pose mapping training data generator 540, and a machine learning engine 530.

FIG. 5B is a flowchart of an exemplary process of the ASM-pose mapping model generator 220, in accordance with an embodiment of the present teaching. In operation, the user interaction interface 500 renders, at 505, the 3D models 240 of a target organ to the user, which may correspond to a manipulable 3D rendering of the modeled target organ. As discussed herein, the rendering is for facilitating the user to specify user specified probe poses 230. For instance, the user may rotate and translate the rendered 3D organ model when the user determines where to define user specified probe poses. In communicating with the user, when the user interaction interface 500 receives, at 515, a user specified 3D probe pose defined with respect to the rendered 3D models 250, it archives that user specified probe pose in 230. In some embodiments, based on a user specified probe pose, the pose-based 2D ASM determiner 520 may create, at 525, additional random 3D probe poses with respect to that user specified 3D probe pose. In other embodiments, the additional 3D probe poses may be generated after all user specified probe poses are specified. With respect to both the user specified and additionally generated probe poses, corresponding 2D slice images may be obtained based on 2D slices (determined based on the 3D models 240 with respect to a perspective associated with each user specified probe pose) and ASMs for such 2D slice images are generated at 535. Each of the ASMs and its corresponding probe pose (either user specified or randomly generated) form a pair or a mapping, i.e., ASM-- >pose.

This process of generating discrete mappings (pairs) for each user specified probe pose continues until the user specified all probe poses. With N discrete mappings created in this manner (e.g., N=k×M, where k is the number of user specified probe poses and M is the number of random probe poses generated for each user specified probe pose), the ASM-pose mapping training data generator 540 creates, at 555, training data 550, which is then used by the machine learning engine 530 to train, at 565, the ASM-pose mapping model to derive, at 575, the trained ASM-pose mapping model 240. As discussed herein, through machine learning, the trained ASM-pose mapping model 240 learns the relationship between 2D ASMs of the 3D anatomical structures at a 3D USPP. Creating additional probe poses with respect to each user specified probe pose may be presented herein as an exemplary way to create more training data. It is described merely for illustration rather than as a limitation. Other schemes to generate random probe poses based on user specified probe poses may also be used. For example, as illustrated herein in FIG. 3C, random probe poses may also be generated within a 3D region (e.g., a sphere) defined with respect to all user specified probe poses according to some pre-set criterion. As another example, a certain density may be specified as a criterion to ensure that an adequate number of additional probe poses will be provided. Any other generation schemes may also be implemented, and they are all within the scope of the present teaching.

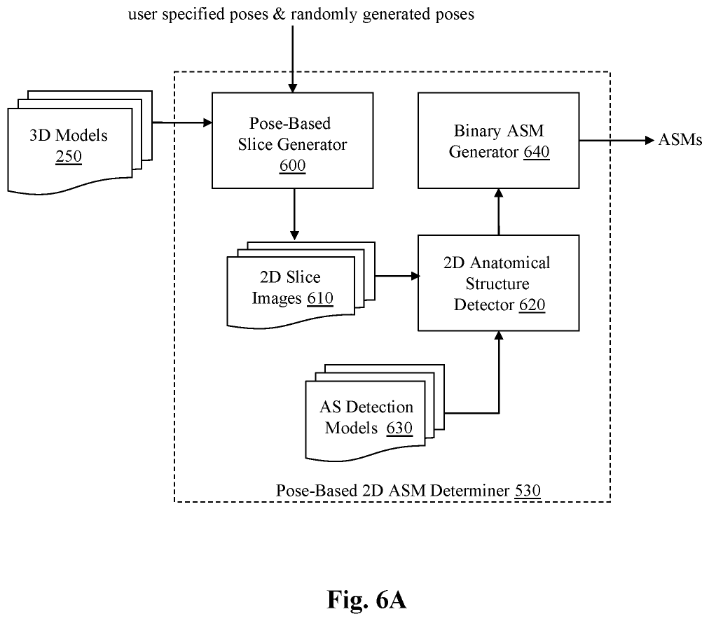

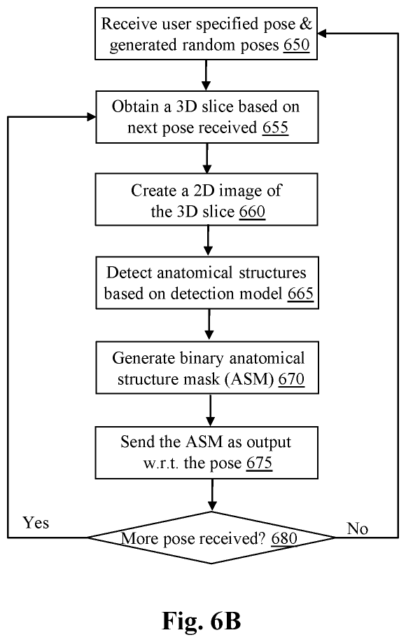

FIG. 6A depicts an exemplary high level system diagram of the pose-based 2D ASM determiner 530, in accordance with an embodiment of the present teaching. As discussed herein, the pose-based 2D ASM determiner 530 is provided to transform each given probe pose (either user specified or randomly generated) into an ASM, i.e., taking a 3D probe pose as an input and producing an ASM as output as an ASM-->3D USPP mapping. In this illustrated embodiment, the pose-based ASM determiner 530 comprises a pose-based slice generator 600, a 2D anatomical structure detector 620, and a binary ASM generator 640. The pose-based slice generator 600 is provided to simulate the process of acquiring an ultrasound image by an ultrasound probe at a given pose. Given an input probe pose, the pose-based slice generator 600 determines a plane in the 3D models 240, sliced according to the input probe pose as well as some operational parameters of the ultrasound probe (e.g., the distance it can transmit the sound wave). In some embodiments, the input probe pose may be used to determine a perspective with respect to the 3D models and the slice is perpendicular to that perspective and is at a distance determined based on the operational parameters of the ultrasound probe. Based on the slice, the pose-based slice generator 600 simulates an ultrasound sensing process to acquire a 2D ultrasound image with anatomical structures therein as what would be sensed by the actual ultrasound probe from the 3D models. Such 2D simulated ultrasound images created based on given probe poses are stored in 610. The 2D anatomical structure detector 620 is provided for detecting anatomical structures in each of the 2D slice images. Such anatomical structures may include tumor(s) and blood vessels. The detection may be carried out based on relevant AS detection models 630. Based on the detected anatomical structures, the binary ASM generator 640 creates corresponding ASMs as output.

FIG. 6B is a flowchart of an exemplary process of the pose-based 2D ASM determiner 530, in accordance with an embodiment of the present teaching. In operation, when the pose-based slice generator 600 receives, at 650, input probe poses (user specified and additionally generated) and obtains, at 655, a 3D slice for each of the input poses in order to create, at 660, a simulated 2D ultrasound image by determining what is visible on the 3D slice. For each of the simulated 2D ultrasound images so created, the 2D anatomical structure detector 620 detects, at 665, anatomical structures appearing in the simulated 2D image. Such detected anatomical structures in a 2D image are used by the binary ASM generator 640 to generate, at 670, an ASM and sends, at 675, the ASM with the input probe pose as a pair as an output. This process of generating a simulated 2D ultrasound image, detecting anatomical structures therefrom, and creating an ASM continues until all ASM-pose mappings are generated for the input probe poses (both user specified and additionally generated), determined at 680.

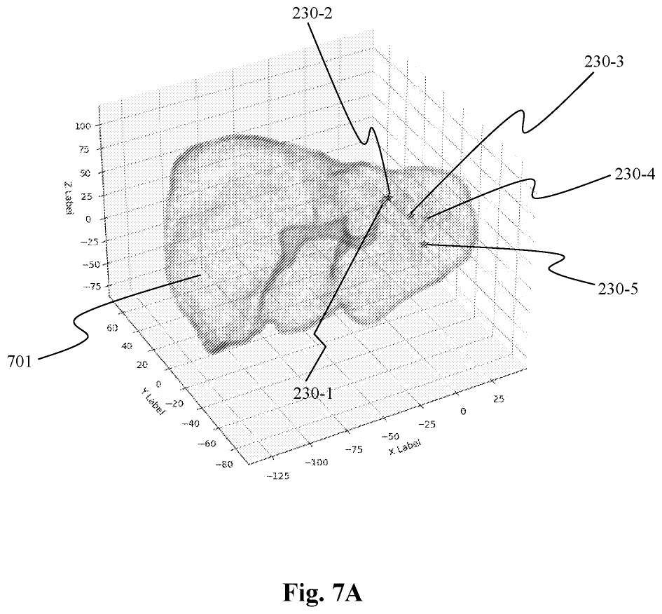

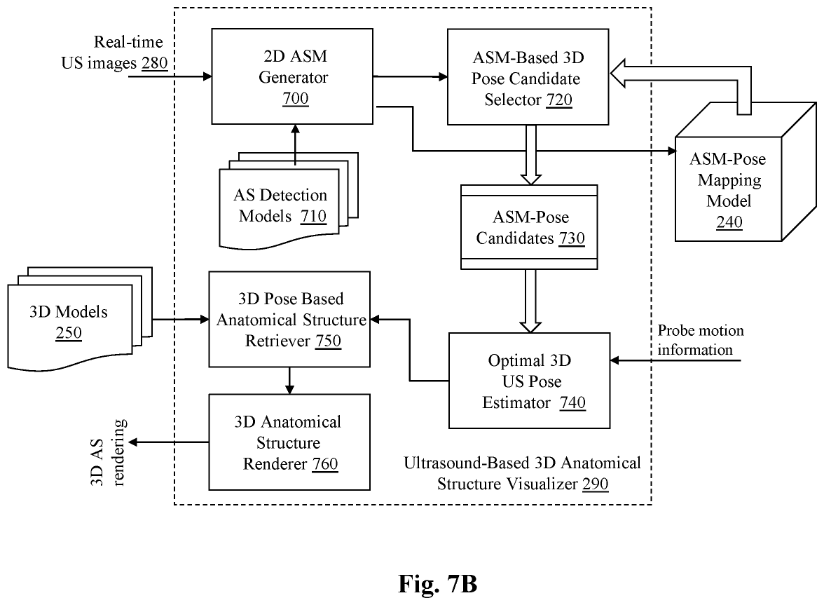

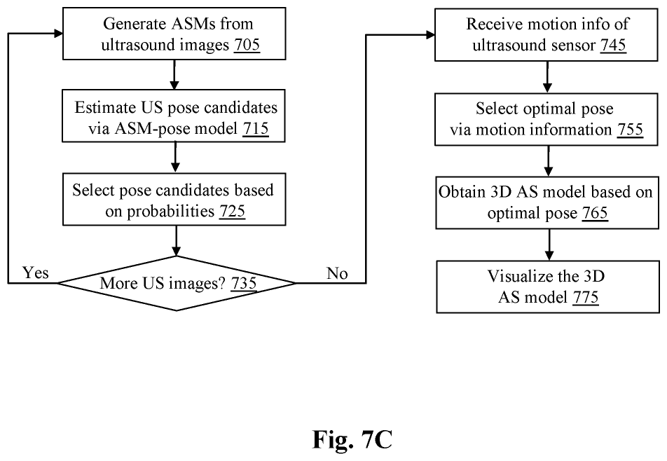

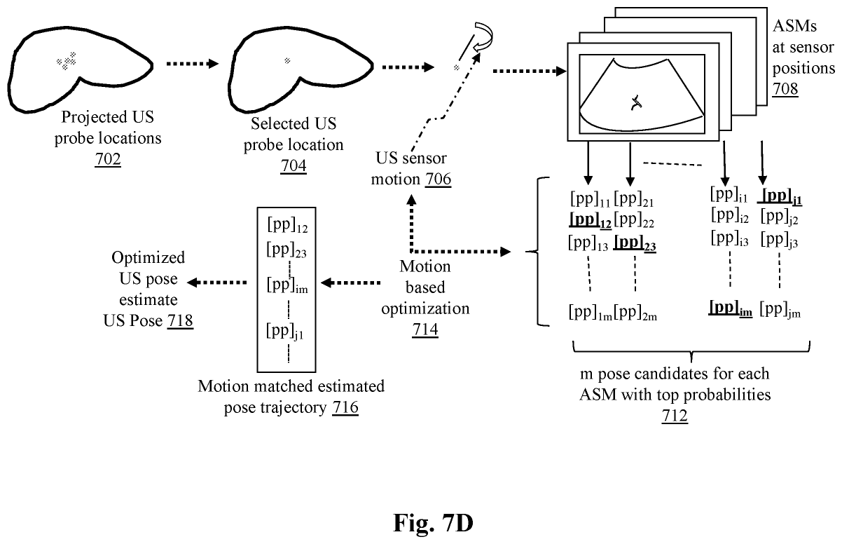

As discussed herein, the pre-surgery part of the 3D USPP based AS visualization system 200 is for creating user specified probe poses 230 and a trained ASM-pose mapping model 240, which are used in the in-surgery phase to predict the accurate 3D USPP of an ultrasound probe. The accurately predicted 3D USPP enables improved visualization of anatomical structures captured in real time ultrasound images. In some embodiments, the 3D USPP may be determined based on multiple candidate 3D USPPs, predicted using the ASM-pose mapping model 240. As disclosed herein, during a surgery, the US probe pose projector 260 first retrieves the user specified probe poses and projects them onto laparoscopic images to facilitate a user to select an initial pose to place an ultrasound probe. FIG. 7A illustrates exemplary projections of the user specified probe poses 230. As shown, 701 may correspond to an organ as captured by a laparoscopic camera. Previously user specified probe poses 230 may then be projected thereon, such as 230-1, 230-2, . . . , 230-5. These projected probe poses in a 2D image may provide alternative poses that a user may select to place an ultrasound probe during the surgery. For example, a user may like to place the ultrasound probe at a pose to see, e.g., a tumor and associated vessel structures beneath the surface of a certain part of the organ 701. When the user directs the ultrasound probe to be deployed at one of the projected locations, real time ultrasound images may be acquired by the ultrasound probe and sent to the ultrasound-based 3D anatomical structure visualizer 290, which may process the real time ultrasound image and estimate the actual 3D USPP of the ultrasound probe so that 3D anatomical structures corresponding to what is seen in the 2D ultrasound images may be visualized to the user to provide enhanced quality.

FIG. 7B depicts an exemplary high level system diagram of the ultrasound-based 3D anatomical structure visualizer 290, in accordance with an embodiment of the present teaching. The input to the ultrasound-based 3D anatomical structure visualizer 290 includes real time ultrasound images 380 acquired by the ultrasound probe as well as location and motion of the ultrasound probe. Its output includes a 3D anatomical structure visualization result and optionally instructions to the user on introducing certain motion of the ultrasound probe. In this illustrated embodiment, the ultrasound-based 3D anatomical structure visualizer 290 comprises a 2D ASM generator 700, an ASM-based 3D pose candidate selector 720, an optimal 3D US pose estimator 740, a 3D pose-based anatomical structure retriever 750, and a 3D anatomical structure renderer 760.

The 2D ASM generator 700 is provided for generating a binarized image according to anatomical structure(s) detected from real time ultrasound images based on, e.g., AS detection models 710. Such generated ASM is sent to the ASM-pose mapping model 240 as input so that the ASM-pose mapping models 240 predicts a plurality of candidate 3D USPPs with respective probabilities as confidence scores of the predictions. The output of the ASM-pose mapping model 240 is provided to the ASM-based 3D pose candidate selector 720 for selecting an appropriate set of, e.g., promising candidate ASM-pose mappings 730. While the ultrasound probe undergoes certain motions, it yields continually a series of 2D ultrasound images. When such images are processed, it yields a series of ASMs, each of which is used to produce, by the ASM-pos mapping model 240, a set of candidate ASM-pose mappings and some are selected (by the ASM-based 3D pose candidate selector 720) in 730 for further processing.

These multiple sets of candidate USPPs saved in 730 are used by the optimal 3D US pose estimator 740 to select an optimal 3D USPP estimate. Each set of candidate poses are estimated with respect to one probe pose (with varying probabilities) and multiple sets of candidate poses correspond to multiple probe poses due to, e.g., slight motion of the probe (e.g., rotations of the probe). To determine an optimal USPP estimate based on multiple sets of candidate poses, different optimization schemes may be employed. In some embodiments, a trajectory of the probe due to its motion may be utilized to determine an optimal USPP estimate. For example, if the probe moves in a clockwise movement, candidate poses selected from associated sets of candidate poses may be selected in a manner so that the selected candidate poses also form a clockwise trajectory and the overall confidence scores of the selected candidate poses are maximum. In such situations, the trajectory formed by the candidate poses selected from different sets is consistent with the actual motion trajectory of the probe. Then the optimal USPP estimate may be determined based on such selected candidate poses. For example, one of the selected candidate poses along the consistent trajectory may be chosen as the optimal one. In another example, the selected candidate pose corresponding to the last probe position may be used as the optimal estimate. Other means to determine the optimal estimated pose may also be possible. For instance, a median or centroid of all candidate poses along the consistent trajectory may be used as the optimal estimated pose.

With the optimal 3D USPP estimate from the optimal 3D US probe estimator 740, the 3D pose-based anatomical structure retriever 750 may accordingly identify the 3D anatomical structures from the 3D models 250 according to the perspective defined by the optimal 3D USPP estimate. As the 3D USPP is obtained in an optimal manner, the accordingly determined anatomical structures may also be the most close to what is observed in real time ultrasound images. With that, the 3D anatomical structure renderer 760 may visualize such determined 3D anatomical structures to provide more effective visual guide to the user, i.e., allowing the user to see the 3D anatomical structures that correspond to what is partially visible in the real time ultrasound images. This visualization not only helps the user to view what is beneath the surface of an organ but also with a full rendering of the corresponding anatomical structures registered with the partial information reveled in real time ultrasound images. Thus, this visualization provides effective visual guide to the user in terms of what is where and accordingly determine what is to be done next during the surgery.

FIG. 7C is a flowchart of an exemplary process of the ultrasound-based 3D anatomical structure visualizer 290, in accordance with an embodiment of the present teaching. Upon receiving real time ultrasound images acquired by a ultrasound probe, the 2D ASM generator 700 detects anatomical structures visible in the ultrasound images and generates, at 705, ASMs for the input ultrasound images. Each of the ASMs is provided to the ASM-pose mapping model 240, which predicts or estimates, at 715, a plurality of candidate 3D USPPs with respective probabilities indicative of the confidence in the estimates. The candidate 3D USPPs with probabilities may be output to the ASM-based 3D pose candidate selector 720, which may select, at 725, some candidate 3D USPPs based on, e.g., their probabilities. For example, the ASM-based 3D pose candidate selector 720 may select only candidate USPP estimates that have probability higher than a pre-determined threshold (e.g., >0.5). The selected candidate USPPs for different ASMs are stored in 730. The processing at steps 705-725 may be continuous so long as the ultrasound pose is still moving or until the user signals to stop. This is determined at 735.

The selected candidate USPP estimates stored in 730 may then be used for optimization to determine an optimal 3D USPP. As discussed herein, there may be multiple sets of candidate USPP estimates stored in 730 and an optimal one may be selected by leveraging the motion information associated with the probe. In some embodiments, the optimal 3D US pose estimator 740 receives, at 745, the probe motion information and selects, at 755, an optimal USPP estimate based on the probe motion information. As discussed above, this may be carried out in two phases. In the first phase, a candidate USPP estimate may be selected from each set of candidates corresponding to each probe pose so that the trajectory formed by these selected candidate USPP estimates form a motion trajectory that is consistent with the motion trajectory of the probe so that the overall probability along the trajectory is maximized. The second phase is to determine an optimal USPP estimate based on the selected candidate USPP estimates. In some embodiments, one of the candidate USPP estimates may be chosen as the optimal USPP estimate, e.g., an estimated USPP corresponding to the last ASM. In other embodiments, the optimal USPP estimate may be derived based on all selected candidate USPP estimates by, e.g., combining them in some fashion to yield an optimal estimated pose, e.g., a centroid of all the selected candidate USPP estimates may be computed as the optimal estimated pose.

Once the optimal USPP estimate is derived, the 3D pose based anatomical structure retriever 750 obtains, at 765, a 3D sub-model from the 3D models 250 according to a perspective determined based on the optimal 3D USPP estimate. The 3D sub-model corresponds to a part of the 3D models 250 and it includes relevant 3D anatomical structures as seen (e.g., partially) in at least some of the real time ultrasound images. Such determined relevant 3D anatomical structures may then be provided to the 3D anatomical structure renderer 760 so that they can be visualized, at 775, to the user. The visualization provides a view of 3D anatomical structures in a way that they are registered with what is revealed in real time ultrasound images and allow the user to see what is inside the target organ, assisting a surgeon to understand the local internal environment in order to make a decision as to what to do and how to do surgical operation.

FIG. 7D provides an exemplary flow of the in-surgery operation during an LUS procedure to estimate a 3D USPP based on 2D ultrasound images, in accordance with an embodiment of the present teaching. This exemplary flow starts from the upper left corner with the operation step 702 that projects user specified probe poses (230) onto 2D laparoscopic images (at projected 2D locations). Based on the projected 2D probe locations, a user (e.g., a surgeon) may select one of the projected 2D locations to place an ultrasound probe at or near the selected probe location (704). The user may also move around the probe (706) according to, e.g., instructions such as “turn your probe clockwise.” The placement of the ultrasound probe and the subsequent motions of the probe produce a series of real time ultrasound images, from which corresponding ASMs are generated (708). Each of the ASMs may then be sent to the SM-pose mapping model 240 and a plurality of candidate 3D USPPs are estimated with respective probabilities. Optionally, the outputs from the ASM-pose mapping model 240 may be filtered to retain those candidate 3D USPPs that meet some pre-determined criterion (712). To determine an optimized 3D USPP estimate based on the candidate 3D USPPs (712), the motion information associated with the ultrasound probe is utilized to identify from 712 those estimated 3D USPPs that form a trajectory consistent with the ultrasound probe motion (714) with maximized probability (716). That is, the candidate 3D USPP estimates form a trajectory consistent with the motion trajectory of the ultrasound probe. Based on the candidate 3D USPP estimates in 716, one optimal one is generated (718) via, e.g., selection of one or combining to create an optimal estimated pose.

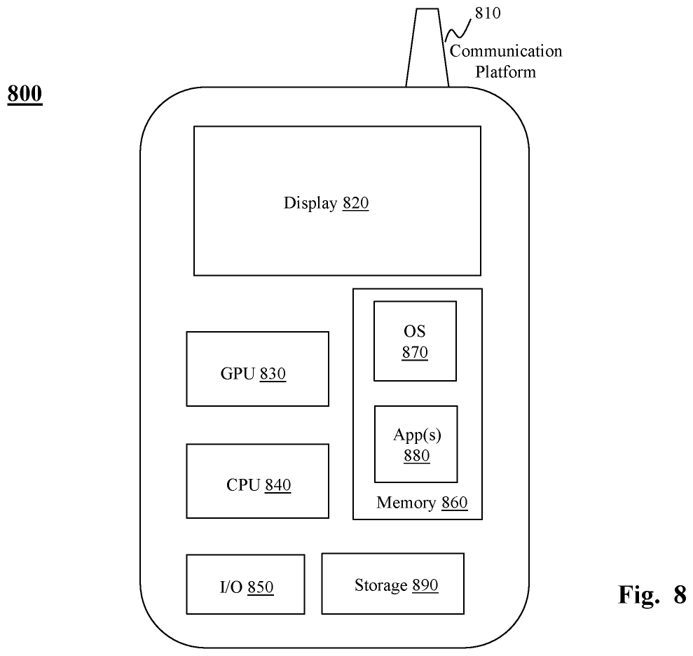

FIG. 8 is an illustrative diagram of an exemplary mobile device architecture that may be used to realize a specialized system implementing the present teaching in accordance with various embodiments. In this example, the user device on which the present teaching may be implemented corresponds to a mobile device 800, including, but not limited to, a smart phone, a tablet, a music player, a handled gaming console, a global positioning system (GPS) receiver, and a wearable computing device, or in any other form factor. Mobile device 800 may include one or more central processing units (“CPUs”) 840, one or more graphic processing units (“GPUs”) 830, a display 820, a memory 860, a communication platform 810, such as a wireless communication module, storage 890, and one or more input/output (I/O) devices 850. Any other suitable component, including but not limited to a system bus or a controller (not shown), may also be included in the mobile device 800. As shown in FIG. 8, a mobile operating system 870 (e.g., iOS, Android, Windows Phone, etc.), and one or more applications 880 may be loaded into memory 860 from storage 890 in order to be executed by the CPU 840. The applications 880 may include a user interface or any other suitable mobile apps for information analytics and management according to the present teaching on, at least partially, the mobile device 800. User interactions, if any, may be achieved via the I/O devices 850 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

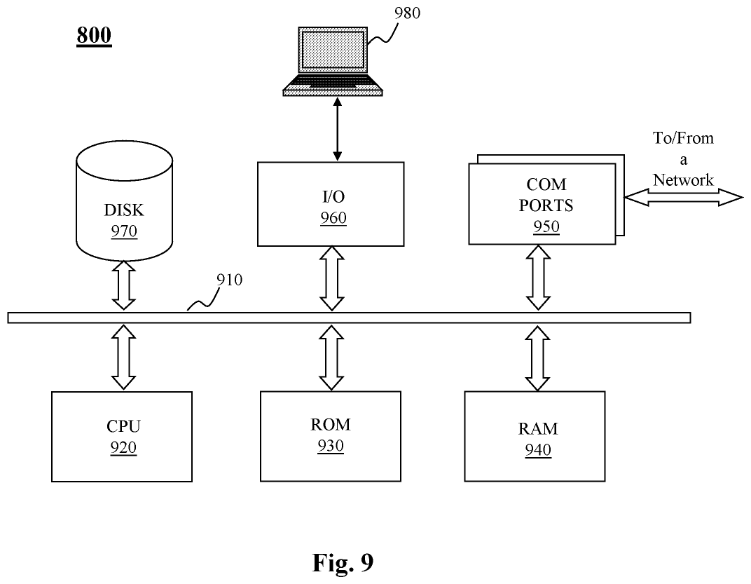

FIG. 9 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 900, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 900, for example, includes COM ports 950 connected to and from a network connected thereto to facilitate data communications. Computer 900 also includes a central processing unit (CPU) 920, in the form of one or more processors, for executing program instructions. The exemplary computer platform includes an internal communication bus 910, program storage and data storage of different forms (e.g., disk 970, read only memory (ROM) 930, or random-access memory (RAM) 940), for various data files to be processed and/or communicated by computer 900, as well as possibly program instructions to be executed by CPU 920. Computer 900 also includes an I/O component 960, supporting input/output flows between the computer and other components therein such as user interface elements 980. Computer 900 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method implemented on at least one processor, a memory, and a communication platform, comprising:
projecting, during a surgery, one or more three-dimensional (3D) probe poses onto a two-dimensional (2D) image at corresponding 2D probe locations, wherein the 2D image is acquired by a laparoscopic camera inserted into a patient's body in the surgery directed to a target organ;
receiving 2D ultrasound images acquired by an ultrasound probe placed at or near one of the 2D probe locations;
with respect to each of at least some of the 2D ultrasound images,
detecting an anatomical structure from the 2D ultrasound image,
generating an anatomical structure mask (ASM) for the 2D ultrasound image based on the detected anatomical structure,
predicting, via an ASM-pose mapping model, one or more 3D probe pose estimates of the ultrasound probe based on the ASM; and
generating an optimal 3D probe pose estimate based on the 3D probe pose estimates predicted based on ASMs obtained from the 2D ultrasound images acquired by the ultrasound probe at varying 3D probe poses.

2. The method of claim 1, wherein the one or more 3D probe poses are specified by a user with respect to a 3D model of the target organ rendered to the user; and
used for creating the ASM-pose mapping model based on ASMs obtained with respect the one or more 3D probe poses.

3. The method of claim 1, wherein each of the one or more 3D probe pose estimates is predicted with a metric indicative of a confidence level of the 3D probe pose estimate.

4. The method of claim 3, wherein the varying 3D probe poses are due to motions of the ultrasound probe around the selected 2D probe location, wherein the motions include variations in terms of 3D coordinate or 3D orientation of the ultrasound probe.

5. The method of claim 4, wherein the step of generating an optimal 3D probe pose estimate comprises:
determining a trajectory of the ultrasound probe according to the varying 3D probe poses;
identifying 3D probe pose estimates predicted with respect to each of the varying 3D probe poses along the trajectory;
determining the optimal 3D probe pose estimate based on the 3D probe pose estimates predicted with respect to the varying 3D probe poses along the trajectory.

6. The method of claim 5, wherein the step of determining the optimal 3D probe pose estimate comprises:
selecting one of the 3D probe pose estimates with respect to each of the varying probe poses along the trajectory so that the metrics of the selected predicted 3D probe poses are maximized; and
determining the optimal 3D probe pose estimate based on the selected predicted 3D probe poses along the trajectory.

7. The method of claim 1, wherein the ASM-pose mapping model is obtained by:
rendering, to a user prior to the surgery, a three-dimensional (3D) model constructed to model the target organ;
receiving instructions from the user specifying the plurality of 3D probe poses;
obtaining simulated 3D probe poses including the user specified plurality of 3D probe poses and the additional 3D probe poses automatically generated with respect to the plurality of 3D probe poses; and
creating the ASM-pose mapping model based on the simulated 3D probe poses.

8. The method of claim 7, wherein the additional 3D probe poses are generated randomly within a 3D region defined based on one or more of the plurality of 3D probe poses.

9. The method of claim 7, wherein the step of creating the ASM-pose mapping model comprises:
with respect to each of the simulated 3D probe poses:
determining a perspective based on the simulated 3D probe pose with respect to the rendered 3D model,
obtaining a simulated two-dimensional (2D) ultrasound image as acquired from the perspective by a simulated ultrasound probe placed at the simulated 3D probe pose,
deriving an anatomical structure mask (ASM) based on anatomical structures in the simulated 2D ultrasound image, and
creating a simulated mapping pair involving the ASM and the simulated 3D probe pose; and
obtaining the ASM-pose mapping model based on the simulated mapping pairs.

10. The method of claim 9, wherein the step of obtaining the ASM-pose mapping model comprises:
generating training data for machine learning based on the simulated mapping pairs;
training, using the training data, the ASM-pose mapping model.

11. A machine readable and non-transitory medium having information recorded thereon, wherein the information, when read by the machine, causes the machine to perform the following steps:
projecting, during a surgery, one or more three-dimensional (3D) probe poses onto a two-dimensional (2D) image at corresponding 2D probe locations, wherein the 2D image is acquired by a laparoscopic camera inserted into a patient's body in the surgery directed to a target organ;
receiving 2D ultrasound images acquired by an ultrasound probe placed at or near one of the 2D probe locations;
with respect to each of at least some of the 2D ultrasound images,
detecting an anatomical structure from the 2D ultrasound image,
generating an anatomical structure mask (ASM) for the 2D ultrasound image based on the detected anatomical structure,
predicting, via an ASM-pose mapping model, one or more 3D probe pose estimates of the ultrasound probe based on the ASM; and
generating an optimal 3D probe pose estimate based on the 3D probe pose estimates predicted based on ASMs obtained from the 2D ultrasound images acquired by the ultrasound probe at varying 3D probe poses.

12. The medium of claim 11, wherein the one or more 3D probe poses are
specified by a user with respect to a 3D model of the target organ rendered to the user; and
used for creating the ASM-pose mapping model based on ASMs obtained with respect the one or more 3D probe poses.

13. The medium of claim 11, wherein each of the one or more 3D probe pose estimates is predicted with a metric indicative of a confidence level of the 3D probe pose estimate.

14. The medium of claim 13, wherein the varying 3D probe poses are due to motions of the ultrasound probe around the selected 2D probe location, wherein the motions include variations in terms of 3D coordinate or 3D orientation of the ultrasound probe.

15. The medium of claim 14, wherein the step of generating an optimal 3D probe pose estimate comprises:
determining a trajectory of the ultrasound probe according to the varying 3D probe poses;
identifying 3D probe pose estimates predicted with respect to each of the varying 3D probe poses along the trajectory;
determining the optimal 3D probe pose estimate based on the 3D probe pose estimates predicted with respect to the varying 3D probe poses along the trajectory.

16. The medium of claim 15, wherein the step of determining the optimal 3D probe pose estimate comprises:
selecting one of the 3D probe pose estimates with respect to each of the varying probe poses along the trajectory so that the metrics of the selected predicted 3D probe poses are maximized; and
determining the optimal 3D probe pose estimate based on the selected predicted 3D probe poses along the trajectory.

17. The medium of claim 11, wherein the ASM-pose mapping model is obtained by:
rendering, to a user prior to the surgery, a three-dimensional (3D) model constructed to model the target organ;
receiving instructions from the user specifying the plurality of 3D probe poses;
obtaining simulated 3D probe poses including the user specified plurality of 3D probe poses and the additional 3D probe poses automatically generated with respect to the plurality of 3D probe poses; and
creating the ASM-pose mapping model based on the simulated 3D probe poses.

18. The medium of claim 17, wherein the additional 3D probe poses are generated randomly within a 3D region defined based on one or more of the plurality of 3D probe poses.

19. The medium of claim 17, wherein the step of creating the ASM-pose mapping model comprises:
with respect to each of the simulated 3D probe poses:
determining a perspective based on the simulated 3D probe pose with respect to the rendered 3D model,
obtaining a simulated two-dimensional (2D) ultrasound image as acquired from the perspective by a simulated ultrasound probe placed at the simulated 3D probe pose,
deriving an anatomical structure mask (ASM) based on anatomical structures in the simulated 2D ultrasound image, and
creating a simulated mapping pair involving the ASM and the simulated 3D probe pose; and
obtaining the ASM-pose mapping model based on the simulated mapping pairs.

20. The medium of claim 19, wherein the step of obtaining the ASM-pose mapping model comprises:
generating training data for machine learning based on the simulated mapping pairs;
training, using the training data, the ASM-pose mapping model.

21. A system, comprising:
an ultrasound probe pose projector implemented by a process and configured for projecting, during a surgery, one or more three-dimensional (3D) probe poses onto a two-dimensional (2D) image at corresponding 2D probe locations, wherein the 2D image is acquired by a laparoscopic camera inserted into a patient's body in the surgery directed to a target organ; and
an ultrasound-based 3D anatomical structure visualizer implemented by a processor and configured for
receiving 2D ultrasound images acquired by an ultrasound probe placed at or near one of the 2D probe locations,
with respect to each of at least some of the 2D ultrasound images,
detecting an anatomical structure from the 2D ultrasound image,
generating an anatomical structure mask (ASM) for the 2D ultrasound image based on the detected anatomical structure,
predicting, via an ASM-pose mapping model, one or more 3D probe pose estimates of the ultrasound probe based on the ASM, and
generating an optimal 3D probe pose estimate based on the 3D probe pose estimates predicted based on ASMs obtained from the 2D ultrasound images acquired by the ultrasound probe at varying 3D probe poses.

22. The system of claim 21, wherein the one or more 3D probe poses are
specified by a user with respect to a 3D model of the target organ rendered to the user; and
used for creating the ASM-pose mapping model based on ASMs obtained with respect the one or more 3D probe poses.

23. The system of claim 21, wherein each of the one or more 3D probe pose estimates is predicted with a metric indicative of a confidence level of the 3D probe pose estimate.

24. The system of claim 23, wherein the varying 3D probe poses are due to motions of the ultrasound probe around the selected 2D probe location, wherein the motions include variations in terms of 3D coordinate or 3D orientation of the ultrasound probe.

25. The system of claim 24, wherein the step of generating an optimal 3D probe pose estimate comprises:
determining a trajectory of the ultrasound probe according to the varying 3D probe poses;
identifying 3D probe pose estimates predicted with respect to each of the varying 3D probe poses along the trajectory;
determining the optimal 3D probe pose estimate based on the 3D probe pose estimates predicted with respect to the varying 3D probe poses along the trajectory.

26. The system of claim 25, wherein the step of determining the optimal 3D probe pose estimate comprises:
selecting one of the 3D probe pose estimates with respect to each of the varying probe poses along the trajectory so that the metrics of the selected predicted 3D probe poses are maximized; and
determining the optimal 3D probe pose estimate based on the selected predicted 3D probe poses along the trajectory.

27. The system of claim 21, further comprising an ASM-pose mapping model generator implemented by a processor and configured for obtaining the ASM-pose mapping model by:
rendering, to a user prior to the surgery, a three-dimensional (3D) model constructed to model the target organ;
receiving instructions from the user specifying the plurality of 3D probe poses;
obtaining simulated 3D probe poses including the user specified plurality of 3D probe poses and the additional 3D probe poses automatically generated with respect to the plurality of 3D probe poses; and
creating the ASM-pose mapping model based on the simulated 3D probe poses.

28. The system of claim 27, wherein the additional 3D probe poses are generated randomly within a 3D region defined based on one or more of the plurality of 3D probe poses.

29. The system of claim 27, wherein the step of creating the ASM-pose mapping model comprises:
with respect to each of the simulated 3D probe poses:
determining a perspective based on the simulated 3D probe pose with respect to the rendered 3D model,
obtaining a simulated two-dimensional (2D) ultrasound image as acquired from the perspective by a simulated ultrasound probe placed at the simulated 3D probe pose,
deriving an anatomical structure mask (ASM) based on anatomical structures in the simulated 2D ultrasound image, and
creating a simulated mapping pair involving the ASM and the simulated 3D probe pose; and
obtaining the ASM-pose mapping model based on the simulated mapping pairs.

30. The system of claim 29, wherein the step of obtaining the ASM-pose mapping model comprises:
generating training data for machine learning based on the simulated mapping pairs;
training, using the training data, the ASM-pose mapping model.
